(19) United States
(12) Patent Application Publication
(10) Pub. No.: US 2024/0082277 A1
(43) Pub. Date: Mar. 14, 2024

(54) MODIFIED ANTIHISTAMINE COMPOSITION AND ANTIVIRAL TREATMENT

(71) Applicant: IMPACT BIOLIFE SCIENCE, INC., Houston, TX (US)

(72) Inventor: Daryl L. THOMPSON, Winter Haven, FL (US)

(21) Appl. No.: 17/890,816

(22) Filed: Aug. 18, 2022

(51) Int. Cl. A61K 31/695; A61K 31/135

(52) U.S. Cl. CPC A61K 31/695; A61K 31/135

(57) ABSTRACT

A composition N,N-dimethyl-2-((methyldiphenylsilyl)oxy)ethan-1-amine having anti-inflammatory properties. The composition is effective for treating various infections including those which have a pro-inflammatory response.

### FIELD OF THE INVENTION

The present invention relates to a unique antihistamine and in particular an antihistamine having anti-inflammatory enhanced efficacy.

### BACKGROUND OF THE INVENTION

Many conventional methods have been proposed to treat currently known viral infections. These include use of known compounds to treat or address symptoms caused by various viruses. Some viruses elicit a pro-inflammatory response. Often a patient succumbs to a viral infection not because of the virus itself but as a result of complications from a pro-inflammatory cascade response which one's body initiates combat the virus. Therefore therapeutic treatments which mitigate an inflammatory response have been proposed and used to treat infections caused by viruses that elicit a pro-inflammatory response.

An example of a virus that causes a pro-inflammatory response is SARS-CoV-2. Accordingly, currently proposed therapeutics include those agents which mitigate a pro-inflammatory response or cascade.

### SUMMARY OF THE INVENTION

The present invention is directed to mitigating a pro-inflammatory cascade response to viral infections. In one form, the present invention is directed to modified antihistamines which have enhanced anti-inflammatory efficacy. As a result, the present modified antihistamine compounds and pharmaceutical compositions limit the occurrence of or reduce the severity of viral infections which include but are not limited to SARS-CoV-2, Ebola, Dengue, Influenza, and Marburg.

The present invention, in one form thereof, is directed to a composition comprising N,N-dimethyl-2-((methyldiphenylsilyl)oxy)ethan-1-amine.

In another form thereof, the present invention is directed to a pharmaceutical composition comprising N,N-dimethyl-2-((methyldiphenylsilyl)oxy)ethan-1-amine.

In another form thereof, the present invention is directed to a composition comprising a compound of Formula I:

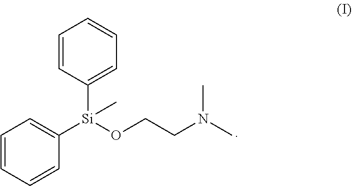 [SMILES: CN(C)CCO[Si](C)(c1ccccc1)c1ccccc1]

The present invention in yet another form thereof is directed to a method of limiting the occurrence or severity of infection from viruses including administering a therapeutically effective amount of N,N-dimethyl-2-((methyldiphenylsilyl)oxy)ethan-1-amine to a patient in need of treatment thereof.

In one further form, the method is directed to an infection caused by viruses that stimulate a cytokine storm. In further alternative forms, the method is directed to viruses selected from the group consisting of SARS-CoV-2, Ebola, Dengue, Influenza, and Marburg.

### BRIEF DESCRIPTION OF THE DRAWINGS

The present invention will now be described with reference to the drawings as follows.

FIG. 1 is a graph relating percent infection to composition concentration.

FIG. 2 is a graph showing relationship between percent cell viability and concentration in accordance with the present invention.

FIG. 3 is a graph relating percent infection to composition concentration.

FIG. 4 is a graph showing relationship between percent cell viability and concentration in accordance with the present invention.

FIG. 5 is a graph relating percent infection to composition concentration.

FIG. 6 is a graph showing relationship between percent cell viability and concentration in accordance with the present invention.

FIG. 7 is a graph relating percent infection to composition concentration.

FIG. 8 is a graph showing relationship between percent cell viability and concentration in accordance with the present invention.

FIG. 9 is a graph relating inhibition with concentration of various antagonists in accordance with the present invention.

FIG. 10 is a graph relating inhibition with diphenhydramine concentration.

FIG. 11 is a graph showing relationship between percent inhibition and diphenhydramine concentration.

FIG. 12 is a graph showing relationship between percent inhibition and concentration of N,N-dimethyl-2-((methyldiphenylsilyl)oxy)ethan-1-amine.

### DETAILED DESCRIPTION

The present invention is directed to a unique composition which includes the compound N,N-dimethyl-2-((methyldiphenylsilyl)oxy)ethan-1-amine.

The compound is synthesized using the following scheme:

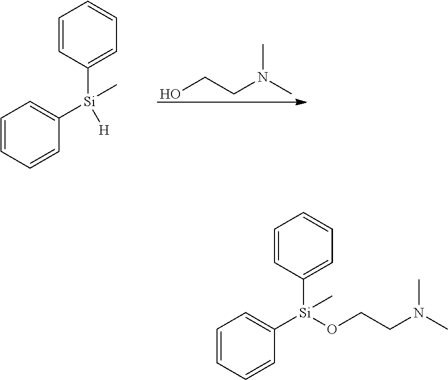

The synthesis uses the following conditions:

| | | EQ | | |
|---|---|---|---|---|
| Solvent | Catalyst | DPMSilane | Time | Result |
| THF | NaOH 10% | 1 | 2 h | TLC showed rapid |
| | | | | production of spot |
| | | | | consistent with |
| | | | | product and less |
| | | | | contaminants, |
| | | | | chromatography |
| | | | | returned colourless |
| | | | | oil, one spot by |
| | | | | TLC and clean |
| | | | | HNMR 370 mg. |

The compound N,N-dimethyl-2-((methyldiphenylsilyl)oxy)ethan-1-amine having the chemical structure of formula (I):

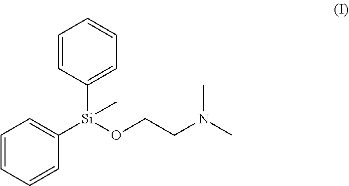 [SMILES: CN(C)CCO[Si](C)(c1ccccc1)c1ccccc1]

This compound is advantageously formulated as a pharmaceutical composition for treating viral infections that have a pro-inflammatory response. These include but are not limited to SARS-CoV-2, Ebola, Dengue, Influenza, and Marburg.

Without being bound to any particular explanation for mechanism of treatment, it is believed that the present pharmaceutical composition may inhibit viral replication and/or treat negative effects of a pro-inflammatory response to various viruses.

Studies conducted demonstrate that the present pharmaceutical composition and compound is successful in downregulating viral replication. Further, N,N-dimethyl-2-((methyldiphenylsilyl)oxy)ethan-1-amine has been shown in this disclosure to be less toxic than traditional antihistamines.

A preferred dose for administration of a composition comprising N,N-dimethyl-2-((methyldiphenylsilyl)oxy)ethan-1-amine in accordance with the present invention is that amount which will be effective in limiting the occurrence of and/or the severity of viral infections especially ones that elicit a pro-inflammatory response, and one would readily recognize that this amount will vary greatly depending on the nature and extent of the disease and the condition of a patient. An “effective amount” of the present compound N,N-dimethyl-2-((methyldiphenylsilyl)oxy)ethan-1-amine to be used (e.g. in a composition including a pharmaceutical composition in accordance with this disclosure) is intended to mean a nontoxic but sufficient amount of the agent, such that the desired prophylactic or therapeutic effect is produced. Thus, the exact amount of N,N-dimethyl-2-((methyldiphenylsilyl)oxy)ethan-1-amine that is required will vary from subject to subject, depending on the species, age, and general condition of the subject, the severity of the condition being treated, the particular carrier or adjuvant being used and its mode of administration, and the like. Similarly, the dosing regimen should also be adjusted to suit the individual to whom the composition is administered and will once again vary with age, weight, metabolism, etc. of the individual. Accordingly, the “effective amount” of any particular composition comprising N,N-dimethyl-2-((methyldiphenylsilyl)oxy)ethan-1-amine will vary based on the particular circumstances, and an appropriate effective amount may be determined in each case of application by one of ordinary skill in the art using only routine experimentation.

### EXPERIMENTS

The present invention will now be described with experiments conducted to demonstrate efficacy of the present composition.

### Experiment 1

Goal:

Evaluate efficacy of antiviral properties of test antibodies against two SARS-CoV-2 variants (delta and omicron).

Method:
1 Vero E6 TMPRSS2 cells were cultured under standard conditions and seeded into 96 well plates until confluent.
2. Compounds were serially diluted half-log down 8 dilutions, using the ranges stated above.
3. Media was removed and compounds added to half of the plates for prophylactic (prior to infection) treatment, according to experimental conditions. These plates were then incubated with the compounds for 1 h before infection.
4. After 1 hour, SARS-CoV-2 was added at a single concentration to all plates. Uninfected plates were also set up.
5. Following infection, overlay media was added to all plates, with equivalent concentrations of compounds added to the previously untreated plates for therapeutic (post infection) treatment.
6. The plates were incubated for 48 hours before being stained with an anti-SARS-CoV-2 nucleocapsid (N) antibody. Cells were imaged to determine percentage infection.
7 Uninfected plates were assessed using a colorimetric MTT assay for cell viability.

Experimental Conditions:

The study was performed in Vero E6 TMPRESS2 overexpressing cells and infected with the following SARS-CoV-2 variants:
Delta (B.1.617.2)
Omicron (B.1.1.529)
   Each experimental condition was tested in triplicate.

Immunostaining and 50% Effective Concentration (EC₅₀)

Presence of SARS-CoV-2 infection was determined by immunostaining with an anti-SARS nucleocapsid (N) antibody at 48 hours post infection. EC₅₀ is the concentration which results in 50% viral infection, following the addition of compounds.

50% Cytotoxic Concentration (CC₅₀)

The concentration which results in 50% cell viability measured using a colorimetric MTT assay, following the addition of compounds.

Selectivity Index (SI)

Calculated as the CC₅₀ divided by the EC₅₀. This is a guide to how selective the compound as towards the virus. Higher SI values indicate promising compounds.

Conditions Table:

| Condition | Dose |
|---|---|
| Q-Ant #8 N,N-dimethyl-2- | 1 mg/ml-0.3 μg/ml |
| ((methyldiphenylsilyl)oxy)ethan-1-amine | |
| Diphenhydramine hydrochloride | 1 mg/ml-0.3 μg/ml |
| WHO standard (pooled convalescent | Dilutions from neat |
| plasma from SARS-CoV-2 patients) | 1 in 50-1 in 15,7318 |
| Remdesivir | 12 μg/ml to 3.8 ng/ml |

Results:

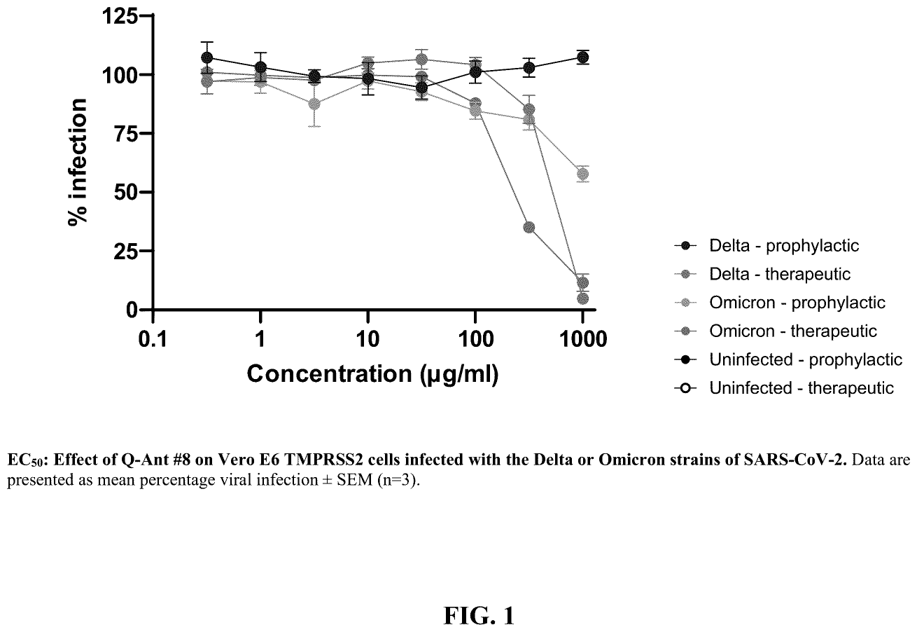

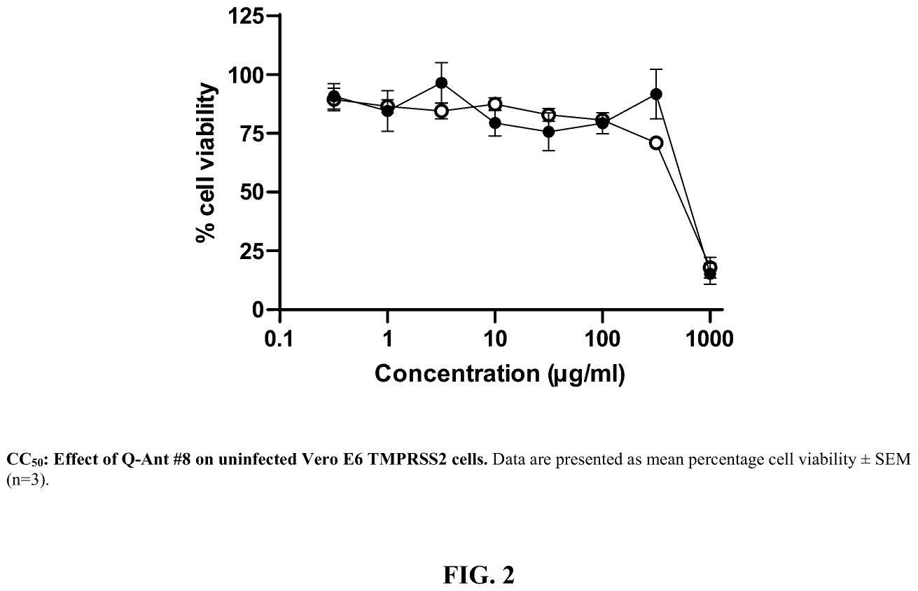

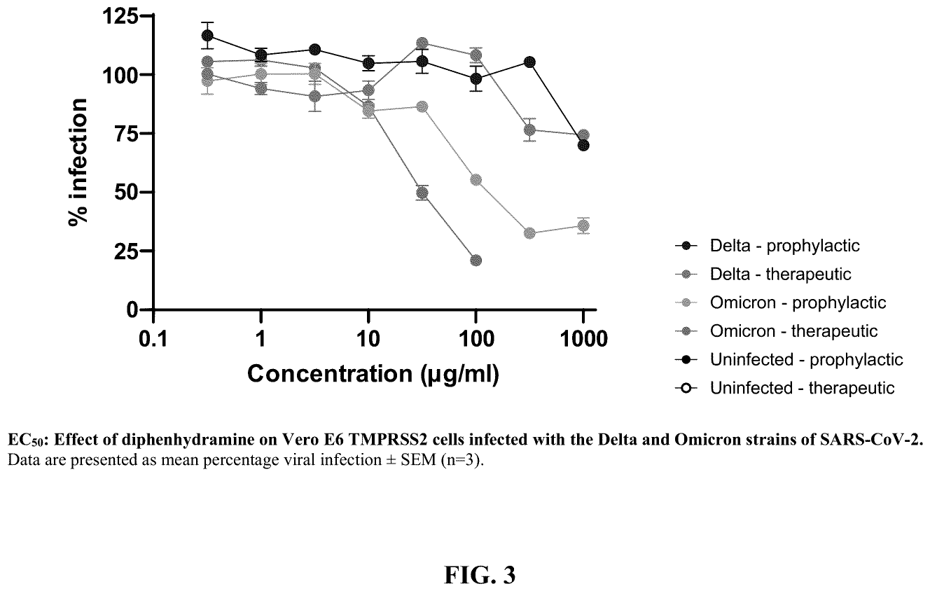

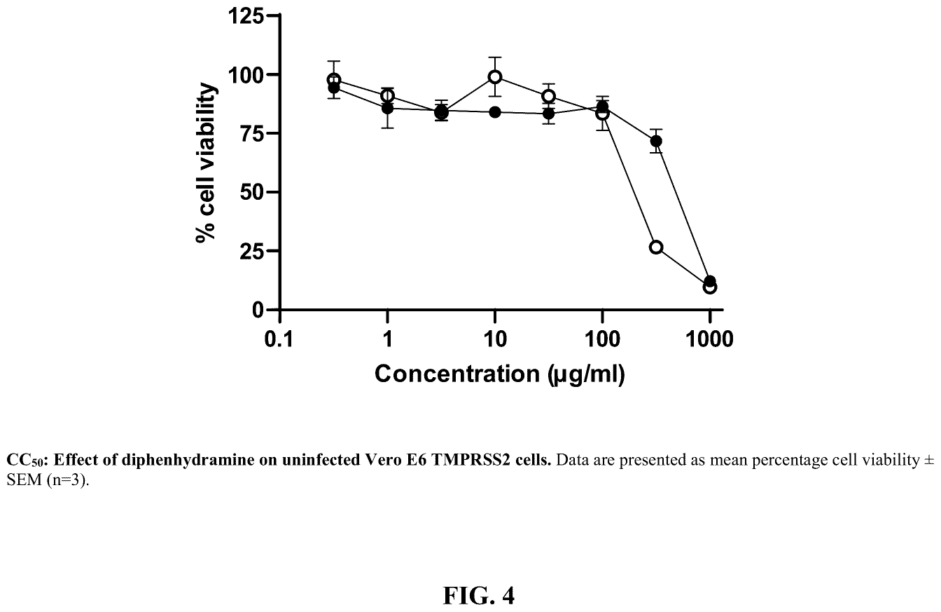

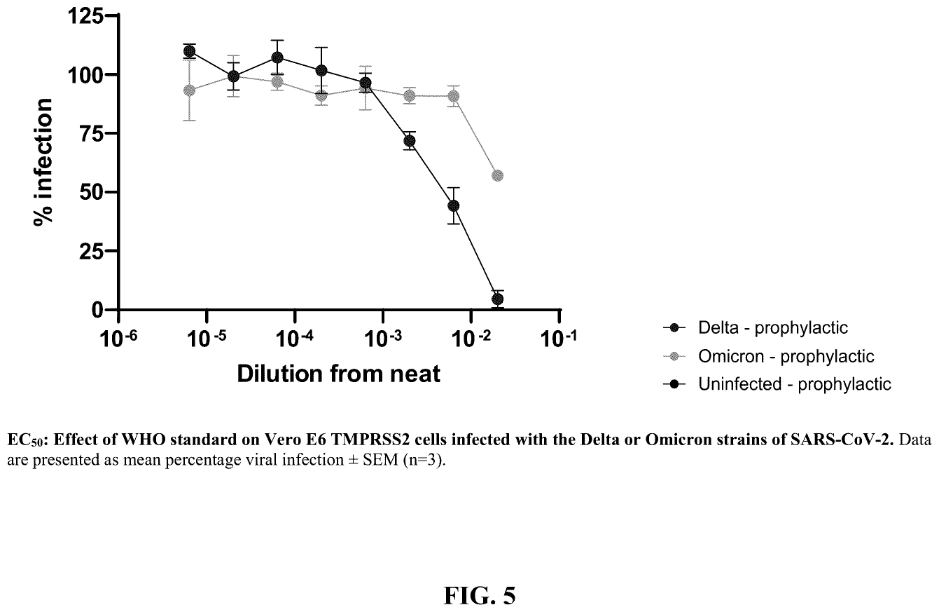

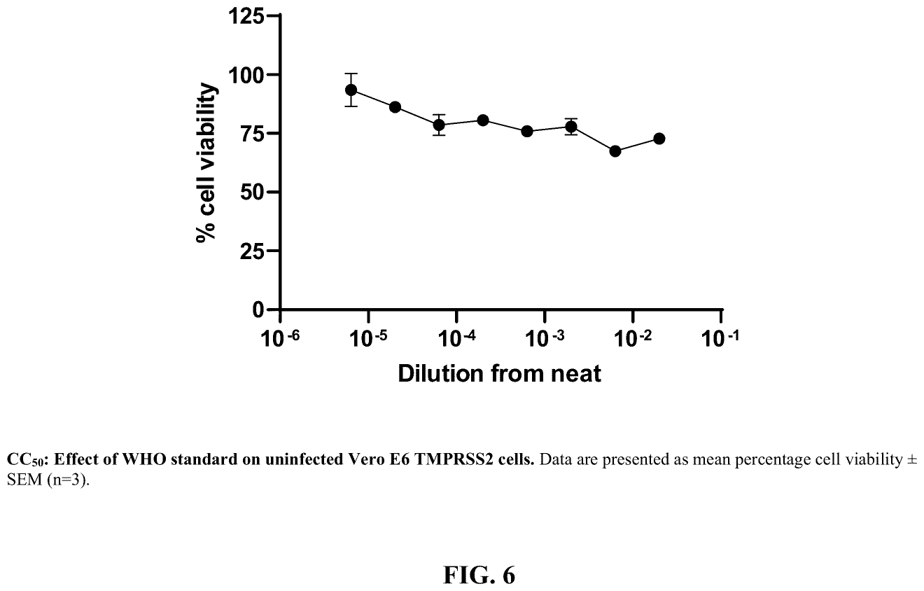

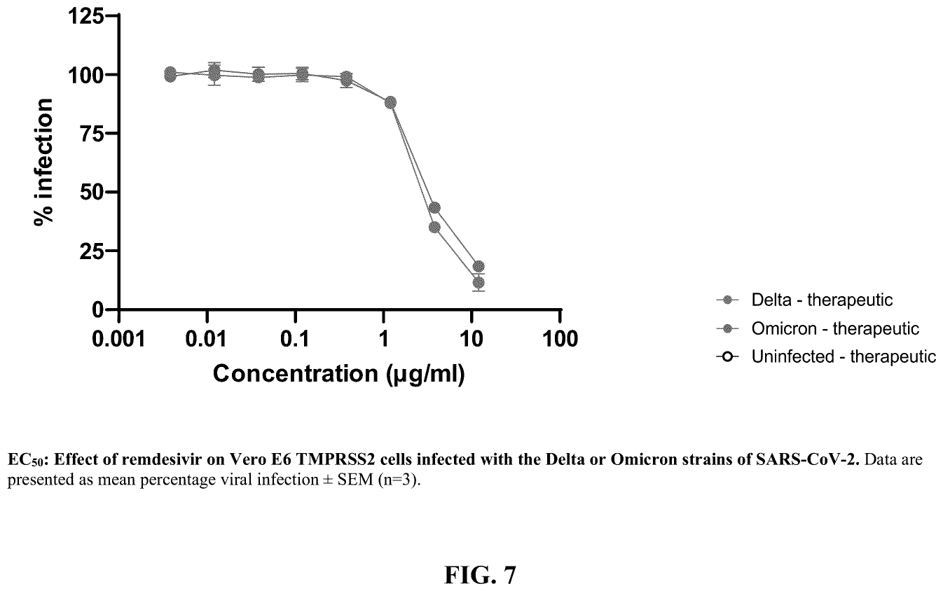

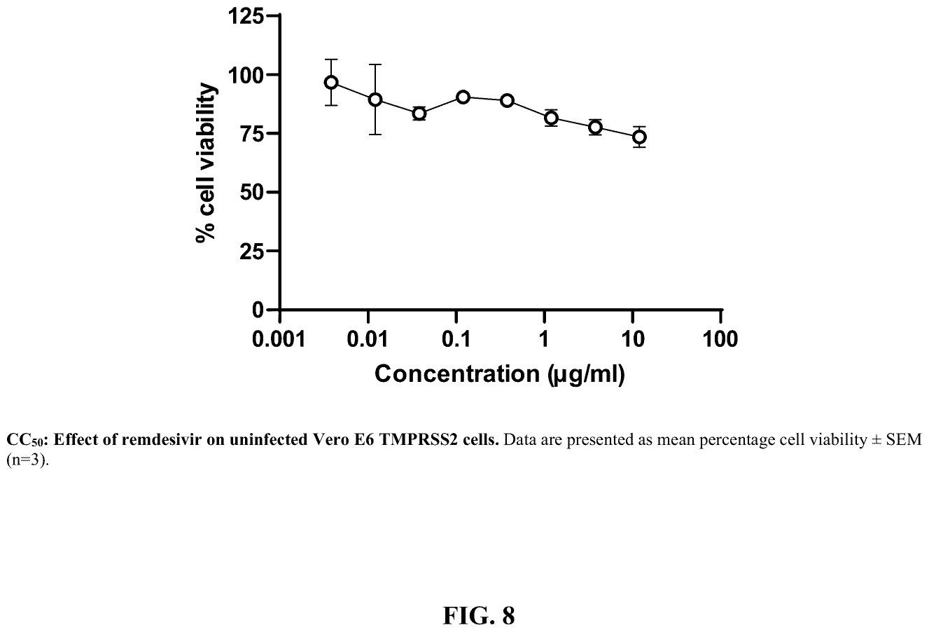

Efficacy results of N,N-dimethyl-2-((methyldiphenylsilyl)oxy)ethan-1-amine are shown in FIGS. 1-8 noting that Q-Ant #8 corresponds to N,N-dimethyl-2-((methyldiphenylsilyl)oxy)ethan-1-amine.

Summary and Conclusions:

Prophylactic treatment

| | | Delta | | Omicron | |
|---|---|---|---|---|---|
| | Cytotoxicity | Efficacy | Selectivity | Efficacy | Selectivity |
| Antibody | (μg/ml) | (μg/ml) | index | (μg/ml) | index |
| Q-Ant #8 N,N-dimethyl-2- | 612.8 | >1000 | N/A | >1000 | N/A |
| ((methyldiphenylsilyl)oxy)ethan- | | | | | |
| 1-amine | | | | | |
| Diphenhydramine | 448.0 | >1000 | N/A | 193.8 | 2.31 |
| WHO standard (dilution from neat) | >0.02 | 0.0046 | >4.35 | >0.02 | N/A |

Therapeutic treatment

| | | Delta | | Omicron | |
|---|---|---|---|---|---|
| | Cytotoxicity | Efficacy | Selectivity | Efficacy | Selectivity |
| Antibody | (μg/ml) | (μg/ml) | index | (μg/ml) | index |
| Q-Ant #8 N,N-dimethyl-2- | 415.3 | 483.0 | 0.86 | 245.6 | 1.69 |
| ((methyldiphenylsilyl)oxy)ethan- | | | | | |
| 1-amine | | | | | |
| Diphenhydramine | 203.1 | >1000 | N/A | 34.4 | 5.90 |
| WHO standard (dilution from neat) | >12 | 2.95 | >4.07 | 3.59 | >3.34 |

Assay controls and positive control drugs worked as expected:
The 50% effective concentration for the WHO standard was around the expected values of 0.004 dilution for Delta and greater than 0.02 dilution for Omicron.
The 50% effective concentration for remdesivir was around the expected value of 3 μg/ml for both Delta and Omicron.

N,N-dimethyl-2-((methyldiphenylsilyl)oxy)ethan-1-amine(Q-Ant #8):
Toxicity was seen following both prophylactic and therapeutic treatment, with CC₅₀ values of 612.8 μg/ml and 415.3 μg/ml, respectively.
Efficacy was seen against both the Delta and Omicron strains of SARS-CoV-2 following therapeutic (post-infection) treatment, with EC₅₀ values of 483.0 μg/ml and 245.6 μg/ml, respectively. The selectivity indices were 0.86 and 1.69, respectively.
A reduction in percentage infection was seen following prophylactic (pre-infection) treatment against the Omicron strain, however this did not reach below 50% at the highest concentration tested. This is encouraging, although not sufficient to calculate efficacy values.
No effect was seen following prophylactic (pre-infection) treatment against the Delta strain.

Diphenhydramine Hydrochloride:
Toxicity was seen following both prophylactic and therapeutic treatment, with CC₅₀ values of 448.0 μg/ml and 203.1 μg/ml, respectively.
Promising efficacy was seen against the Omicron strain following both prophylactic and therapeutic treatment, with EC₅₀ values of 193.8 μg/ml and 34.4 μg/ml, respectively. The selectivity indices were 2.31 and 5.90, respectively.
A slight reduction in percentage infection was seen following prophylactic and therapeutic treatment against the Delta strain, however this did not reach below 50% at the highest concentration tested. This is encouraging, although not sufficient to calculate efficacy values.

### Experiment 2

Summary:

The objective of this study (Experiment 2) was to evaluate the effects in vitro of two (2) test articles on human H1 histamine receptors (HRH1) expressed in CHO-K1 cells using a calcium sensitive dye and a Fluorescence Imaging Plate Reader (FLIPR^{TETRA}™) instrument.

The effects of the test articles were evaluated on HRH1 were evaluated at eight (8) concentrations (0.3, 1, 3, 10, 30, 100, 300 and 1000 nM, n=4). The test article results are presented as normalized % inhibition (antagonist assay). The results are summarized Table 1 and detailed in Table 2. For the test article evaluation, effects were considered significant if the test article mean value was three or more standard deviations below the positive control agonist mean (highlighted in yellow). The threshold values at 3×SD for the assay are listed under Table 2.

TABLE 1
Summary of the Test Articles and Positive
Antagonist Control on HRH1:

| TA # | Test Article ID | IC₅₀, nM |
|---|---|---|
| 1 | Diphenhydramine | 603.4 |
| | Hydrochloride | |
| 2 | Q-Ant N,N-dimethyl-2- | 33.5 |
| | ((methyldiphenylsilyl)oxy)ethan- | |
| | 1-amine. | |
| PC | Cetirizine Dihydrochloride | 2478 |

TABLE 2
Effect of the Test Articles and Positive
Antagonist Control on HRH1 Channels:

| | Test | Normalized | | | |
|---|---|---|---|---|---|
| | Conc. | % | | | IC₅₀, |
| Test Article ID | (nM) | Inhibition | SD | N | (nM) |
| Diphenhydramine | 0.3 | −34.50 | 5.75 | 4 | 603.4 |
| Hydrochloride | 1 | −37.95 | 4.35 | 4 | |
| | 3 | −33.58 | 1.58 | 4 | |
| | 10 | −30.06 | 7.75 | 4 | |
| | 30 | −11.15 | 4.30 | 4 | |
| | 100 | 5.17 | 4.20 | 4 | |
| | 300 | 40.69 | 5.12 | 4 | |
| | 1000 | 82.79 | 1.61 | 4 | |
| Q-Ant #8 N,N-dimethyl- | 0.3 | −32.60 | 2.66 | 4 | 33.5 |
| 2-((methyldiphenyl- | 1 | −33.43 | 0.52 | 4 | |
| silyl)oxy)ethan-1-amine. | 3 | −30.97 | 3.64 | 4 | |
| | 10 | −11.93 | 3.05 | 4 | |
| | 30 | 29.38 | 6.65 | 4 | |
| | 100 | 72.93 | 3.62 | 4 | |
| | 300 | 97.06 | 1.65 | 4 | |
| | 1000 | 97.73 | 1.77 | 4 | |
| Cetirizine | 10 | 23.87 | 2.82 | 4 | 2478 |
| Dihydrochloride | 30 | 23.62 | 3.72 | 4 | |
| | 100 | 15.27 | 4.45 | 4 | |
| | 300 | 14.17 | 3.05 | 4 | |
| | 1000 | 31.26 | 3.63 | 4 | |
| | 3000 | 67.34 | 2.46 | 4 | |
| | 10000 | 98.86 | 0.77 | 4 | |
| | 30000 | 100.00 | 0.54 | 4 | |
| | 100000 | 99.28 | 0.60 | 4 | |

3 × SD of positive agonist (70 nM Histamine) = 24.38; values >24.38 are significant.

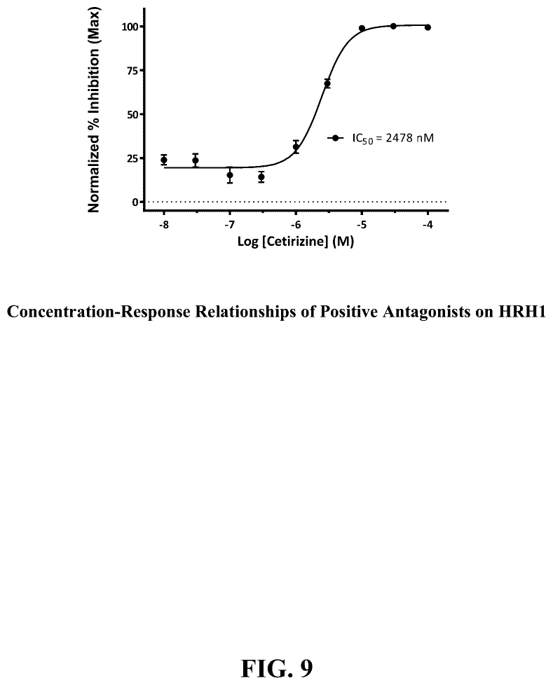

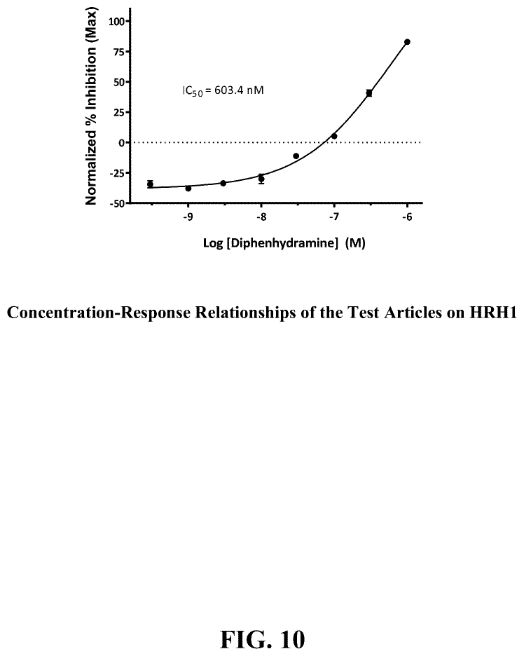

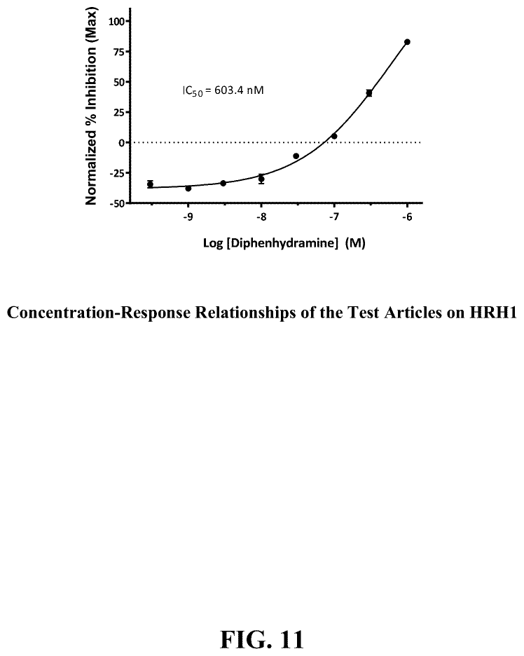

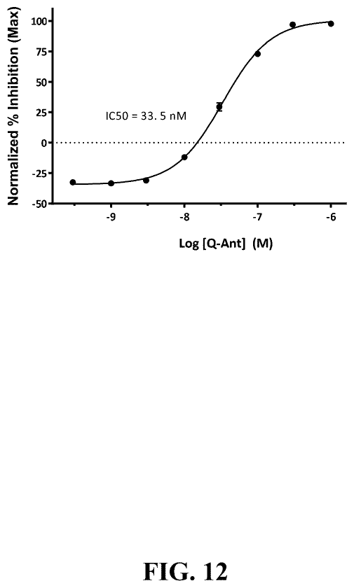

The quality control results (maximal response, minimal response, Z-prime and signal window) for the assay are presented in Table 3. Concentration-response curve for positive control and test articles are presented in FIGS. 9-12. The positive control result confirmed the sensitivity of the test system to the receptor/ion channel inhibition.

The concentration-response curves were fitted in GraphPad Prism 6 using the better fit between a 4-parameter curve (variable slope) and no constraints were set on the curve fits. For the fitting, the data of normalized percent inhibitions were used.

FLIPR Experimental Procedures:

H1 Histamine Receptor Procedures:

The ability of each test article to act as an antagonist of the HRH1 was evaluated in a calcium flux assay (Fluo-8, AAT Bioquest).

For the antagonist effect assessment, the channels were activated with the positive control agonist (70 nM histamine). The effects of the test article to inhibit the signal was examined after agonist stimulation and compared to the respective positive control antagonist (30 μM cetirizine). For the assay, the signal, elicited in the presence of the positive control agonist, was set to 100 (0% inhibition) and the signal in the presence of the positive control antagonist was set to 0 (100% inhibition).

FLIPR Data Tables and Figures:

TABLE 3
Effects of Positive Controls - Plate QC for Antagonist Assays:

| Ion Channel | Control Article | RLU* | SD |
|---|---|---|---|
| HRH1 | 70 nM Histamine | 3431.5 | 266.9 |
| | 30 μM Cetirizine | 147.3 | 17.6 |

Z′ = 0.74; SW = 9.11

*RLU: The maximum Relative Light Unit (Max).

H1 Histamine Receptor Results:

Summary for the test articles and positive controls effects on HRH1 are presented in Table 1. Mean, SD and N numbers for each test article concentration are provided in Table 2.

### Experiment 3

Efficacy of N,N-dimethyl-2-((methyldiphenylsilyl)oxy)ethan-1-amine as prophylactic and as a therapeutic treatment for both SARS-CoV-2, delta variant and omicron variant are shown in the following tables:

Delta Variant:

Prophylactic Treatment - Infected Cells:

| | Concentration | % viral infection | | |
|---|---|---|---|---|
| Compound | (μg/ml) | 1 | 2 | 3 |
| Q-Ant #8 N,N-dimethyl- | 1000.000 | 109.45 | 101.77 | 111.20 |
| 2-((methyldiphenyl- | 316.456 | 102.40 | 96.35 | 110.20 |
| silyl)oxy)ethan-1-amine | 100.144 | 102.70 | 92.17 | 108.33 |
| | 31.691 | 95.02 | 85.80 | 102.84 |
| | 10.029 | 93.45 | 89.47 | 111.99 |
| | 3.174 | 103.97 | 94.61 | 99.31 |
| | 1.004 | 104.92 | 91.82 | 112.81 |
| | 0.318 | 95.44 | 107.71 | 118.49 |
| Diphenhydramine | 1000.000 | 72.42 | 66.55 | 70.91 |
| hydrochloride | 316.456 | 101.20 | 106.00 | 108.79 |
| | 100.144 | 91.85 | 94.25 | 108.88 |
| | 31.691 | 95.89 | 113.08 | 107.99 |
| | 10.029 | 101.45 | 101.85 | 111.14 |
| | 3.174 | 109.52 | 108.14 | 114.49 |
| | 1.004 | 104.63 | 113.98 | 106.58 |
| | 0.318 | 118.09 | 125.52 | 106.26 |
| WHO standard (pooled | 0.020 | 1.71 | 11.78 | 0.18 |
| convalescent plasma | 0.006 | 54.44 | 49.31 | 29.01 |
| from SARS-CoV-2 | 0.002 | 72.92 | 77.88 | 64.54 |
| patients) Dilution | 0.001 | 88.85 | 102.79 | 98.03 |
| from neat | 0.000 | 82.79 | 106.99 | 115.44 |
| | 0.000 | 99.23 | 100.63 | 121.89 |
| | 0.000 | 87.94 | 101.79 | 107.79 |
| | 0.000 | 107.43 | 116.00 | 106.40 |

Delta Variant:

Prophylactic Treatment - Uninfected Cells:

| | Concentration | % cell viability | | |
|---|---|---|---|---|
| Compound | (μg/ml) | 1 | 2 | 3 |
| Q-Ant #8 N,N-dimethyl- | 1000.000 | 10.49 | 24.15 | 11.04 |
| 2-((methyldiphenyl- | 316.456 | 74.27 | 90.37 | 110.50 |
| silyl)oxy)ethan-1-amine | 100.144 | 74.79 | 88.30 | 74.92 |
| | 31.691 | 62.59 | 90.25 | 74.14 |
| | 10.029 | 69.86 | 89.08 | 79.47 |
| | 3.174 | 79.99 | 100.37 | 109.33 |
| | 1.004 | 68.95 | 98.69 | 85.96 |
| | 0.318 | 79.99 | 96.22 | 96.09 |
| Diphenhydramine | 1000.000 | 11.54 | 12.37 | 12.57 |
| hydrochloride | 316.456 | 66.09 | 67.13 | 81.80 |
| | 100.144 | 81.68 | 87.13 | 90.50 |
| | 31.691 | 91.93 | 77.00 | 81.16 |
| | 10.029 | 85.18 | 83.88 | 83.10 |
| | 3.174 | 76.48 | 86.22 | 91.41 |
| | 1.004 | 77.39 | 77.00 | 102.71 |
| | 0.318 | 85.70 | 95.96 | 101.54 |
| WHO standard (pooled | 0.020 | 68.28 | 74.83 | 75.30 |
| convalescent plasma | 0.006 | 71.79 | 65.83 | 64.77 |
| from SARS-CoV-2 | 0.002 | 73.19 | 75.76 | 84.53 |
| patients) Dilution | 0.001 | 76.70 | 72.72 | 78.34 |
| from neat | 0.000 | 80.21 | 77.52 | 84.07 |
| | 0.000 | 70.62 | 85.59 | 79.51 |
| | 0.000 | 84.30 | 85.24 | 88.98 |
| | 0.000 | 81.03 | 105.46 | 94.12 |

Delta Variant:

Therapeutic Treatment - Infected Cells:

| | Concentration | % cell viability | | |
|---|---|---|---|---|
| Compound | (μg/ml) | 1 | 2 | 3 |
| Q-Ant #8 N,N-dimethyl- | 1000.000 | 1.28 | 4.67 | 8.26 |
| 2-((methyldiphenyl- | 316.456 | 73.52 | 90.09 | 92.16 |
| silyl)oxy)ethan-1-amine | 100.144 | 98.17 | 108.67 | 105.86 |
| | 31.691 | 99.37 | 106.02 | 114.03 |
| | 10.029 | 100.03 | 106.30 | 108.44 |
| | 3.174 | 94.95 | 96.37 | 101.62 |
| | 1.004 | 93.94 | 101.39 | 101.14 |
| | 0.318 | 102.20 | 86.58 | 102.33 |
| Diphenhydramine | 1000.000 | 72.76 | 76.58 | 73.55 |
| hydrochloride | 316.456 | 82.77 | 66.98 | 79.71 |
| | 100.144 | 113.51 | 108.74 | 102.52 |
| | 31.691 | 111.08 | 117.47 | 111.92 |
| | 10.029 | 87.59 | 91.51 | 101.19 |
| | 3.174 | 85.55 | 83.22 | 103.59 |
| | 1.004 | 97.34 | 96.10 | 88.82 |
| | 0.318 | 98.78 | 104.42 | 97.53 |
| Remdesivir | 12.000 | 18.60 | 6.48 | 9.57 |
| | 3.797 | 36.79 | 33.95 | 34.45 |
| | 1.202 | 89.80 | 83.56 | 90.29 |
| | 0.380 | 100.87 | 99.72 | 96.88 |
| | 0.120 | 104.76 | 95.04 | 99.85 |
| | 0.038 | 96.83 | 98.54 | 100.93 |
| | 0.012 | 91.34 | 103.01 | 104.89 |
| | 0.004 | 105.27 | 96.93 | 100.76 |

Delta Variant:

Therapeutic Treatment - Uninfected Cells:

| | Concentration | % cell viability | | |
|---|---|---|---|---|
| Compound | (μg/ml) | 1 | 2 | 3 |
| Q-Ant #8 N,N-dimethyl- | 1000.000 | 10.50 | 17.29 | 25.71 |
| 2-((methyldiphenyl- | 316.456 | 67.81 | 75.43 | 69.74 |
| silyl)oxy)ethan-1-amine | 100.144 | 79.30 | 83.62 | 78.96 |
| | 31.691 | 79.64 | 80.66 | 88.40 |
| | 10.029 | 87.15 | 82.94 | 92.38 |
| | 3.174 | 87.38 | 77.93 | 88.40 |
| | 1.004 | 91.70 | 81.69 | 86.24 |
| | 0.318 | 89.20 | 81.23 | 97.96 |
| Diphenhydramine | 1000.000 | 9.86 | 10.50 | 8.54 |
| hydrochloride | 316.456 | 28.78 | 28.10 | 23.10 |
| | 100.144 | 85.78 | 94.55 | 70.08 |
| | 31.691 | 80.44 | 97.16 | 94.77 |
| | 10.029 | 111.50 | 102.62 | 83.05 |
| | 3.174 | 77.37 | 85.78 | 88.63 |
| | 1.004 | 97.50 | 89.08 | 86.24 |
| | 0.318 | 88.06 | 91.59 | 113.55 |
| Remdesivir | 12.000 | 65.24 | 74.83 | 80.32 |
| | 3.797 | 74.95 | 73.89 | 84.18 |
| | 1.202 | 79.39 | 77.05 | 88.39 |
| | 0.380 | 88.04 | 87.92 | 91.20 |
| | 0.120 | 93.07 | 89.44 | 88.74 |
| | 0.038 | 84.42 | 87.81 | 78.34 |
| | 0.012 | 63.61 | 115.17 | 89.44 |
| | 0.004 | 77.05 | 106.28 | 106.87 |

Omicron Variant:

Prophylactic Treatment - Infected Cells:

| | Concentration | % viral infection | | |
|---|---|---|---|---|
| Compound | (μg/ml) | 1 | 2 | 3 |
| Q-Ant #8 N,N-dimethyl- | 1000.000 | 55.19 | 64.49 | 53.68 |
| 2-((methyldiphenyl- | 316.456 | 72.65 | 87.74 | 82.37 |
| silyl)oxy)ethan-1-amine | 100.144 | 90.82 | 78.20 | 84.75 |
| | 31.691 | 89.16 | 88.91 | 100.32 |
| | 10.029 | 90.45 | 99.71 | 101.72 |
| | 3.174 | 68.28 | 97.73 | 96.56 |
| | 1.004 | 94.45 | 90.24 | 106.14 |
| | 0.318 | 97.18 | 94.62 | 100.04 |
| Diphenhydramine | 1000.000 | 32.53 | 42.51 | 32.24 |
| hydrochloride | 316.456 | 33.75 | 33.86 | 30.10 |
| | 100.144 | 57.93 | 56.22 | 51.64 |
| | 31.691 | 89.47 | 86.35 | 83.45 |
| | 10.029 | 90.57 | 81.35 | 81.69 |
| | 3.174 | 108.82 | 98.60 | 93.54 |
| | 1.004 | 102.75 | 106.49 | 91.48 |
| | 0.318 | 108.59 | 92.91 | 90.65 |
| WHO standard (pooled | 0.020 | 57.95 | 54.76 | 58.42 |
| convalescent plasma | 0.006 | 86.23 | 86.56 | 99.68 |
| from SARS-CoV-2 | 0.002 | 88.43 | 86.81 | 97.81 |
| patients) Dilution | 0.001 | 75.67 | 103.24 | 103.77 |
| from neat | 0.000 | 99.25 | 86.23 | 87.90 |
| | 0.000 | 89.58 | 101.61 | 99.49 |
| | 0.000 | 82.06 | 110.70 | 105.14 |
| | 0.000 | 70.66 | 114.91 | 94.32 |

Omicron Variant:

Prophylactic Treatment - Uninfected Cells:

| | Concentration | % cell viability | | |
|---|---|---|---|---|
| Compound | (μg/ml) | 1 | 2 | 3 |
| Q-Ant #8 N,N-dimethyl- | 1000.000 | 10.49 | 24.15 | 11.04 |
| 2-((methyldiphenyl- | 316.456 | 74.27 | 90.37 | 110.50 |
| silyl)oxy)ethan-1-amine | 100.144 | 74.79 | 88.30 | 74.92 |
| | 31.691 | 62.59 | 90.25 | 74.14 |
| | 10.029 | 69.86 | 89.08 | 79.47 |
| | 3.174 | 79.99 | 100.37 | 109.33 |
| | 1.004 | 68.95 | 98.69 | 85.96 |
| | 0.318 | 79.99 | 96.22 | 96.09 |
| Diphenhydramine | 1000.000 | 11.54 | 12.37 | 12.57 |
| hydrochloride | 316.456 | 66.09 | 67.13 | 81.80 |
| | 100.144 | 81.68 | 87.13 | 90.50 |
| | 31.691 | 91.93 | 77.00 | 81.16 |
| | 10.029 | 85.18 | 83.88 | 83.10 |
| | 3.174 | 76.48 | 86.22 | 91.41 |
| | 1.004 | 77.39 | 77.00 | 102.71 |
| | 0.318 | 85.70 | 95.96 | 101.54 |
| WHO standard (pooled | 0.020 | 68.28 | 74.83 | 75.30 |
| convalescent plasma | 0.006 | 71.79 | 65.83 | 64.77 |
| from SARS-CoV-2 | 0.002 | 73.19 | 75.76 | 84.53 |
| patients) Dilution | 0.001 | 76.70 | 72.72 | 78.34 |
| from neat | 0.000 | 80.21 | 77.52 | 84.07 |
| | 0.000 | 70.62 | 85.59 | 79.51 |
| | 0.000 | 84.30 | 85.24 | 88.98 |
| | 0.000 | 81.03 | 105.46 | 94.12 |

Omicron Variant:

Therapeutic Treatment - Infected Cells:

| | Concentration | % cell viability | | |
|---|---|---|---|---|
| Compound | (μg/ml) | 1 | 2 | 3 |
| Q-Ant #8 N,N-dimethyl- | 1000.000 | 18.60 | 6.48 | 9.57 |
| 2-((methyldiphenyl- | 316.456 | 36.79 | 33.95 | 34.45 |
| silyl)oxy)ethan-1-amine | 100.144 | 89.80 | 83.56 | 90.29 |
| | 31.691 | 100.87 | 99.72 | 96.88 |
| | 10.029 | 104.76 | 95.04 | 99.85 |
| | 3.174 | 96.83 | 98.54 | 100.93 |
| | 1.004 | 91.34 | 103.01 | 104.89 |
| | 0.318 | 105.27 | 96.93 | 100.76 |
| Diphenhydramine | 1000.000 | 96.55* | 116.54* | 43.16* |
| hydrochloride | 316.456 | 35.08* | 37.26* | 38.1* |
| | 100.144 | 20.63 | 20.10 | 22.38 |
| | 31.691 | 52.22 | 43.56 | 53.37 |
| | 10.029 | 88.15 | 83.31 | 88.18 |
| | 3.174 | 101.91 | 99.74 | 106.70 |
| | 1.004 | 105.21 | 102.23 | 111.23 |
| | 0.318 | 102.70 | 103.98 | 109.89 |
| Remdesivir | 12.000 | 20.68 | 18.05 | 16.50 |
| | 3.797 | 44.60 | 40.31 | 45.17 |
| | 1.202 | 84.09 | 88.83 | 92.30 |
| | 0.380 | 92.80 | 103.08 | 96.43 |
| | 0.120 | 105.03 | 100.40 | 95.94 |
| | 0.038 | 105.52 | 94.88 | 100.11 |
| | 0.012 | 95.93 | 107.39 | 102.31 |
| | 0.004 | 101.27 | 98.31 | 98.20 |

*This is an artificial increase in percentage infection, due the large reduction in the number of viable cells, as shown in the following “Therapeutic Treatment - Uninfected Cells” table. When calculating the 50% effective concentration, these data points were removed from the non-linear regression analysis.

Omicron Variant:

Therapeutic Treatment - Uninfected Cells:

| | Concentration | % cell viability | | |
|---|---|---|---|---|
| Compound | (μg/ml) | 1 | 2 | 3 |
| Q-Ant #8 N,N-dimethyl- | 1000.000 | 10.50 | 17.29 | 25.71 |
| 2-((methyldiphenyl- | 316.456 | 67.81 | 75.43 | 69.74 |
| silyl)oxy)ethan-1-amine | 100.144 | 79.30 | 83.62 | 78.96 |
| | 31.691 | 79.64 | 80.66 | 88.40 |
| | 10.029 | 87.15 | 82.94 | 92.38 |
| | 3.174 | 87.38 | 77.93 | 88.40 |
| | 1.004 | 91.70 | 81.69 | 86.24 |
| | 0.318 | 89.20 | 81.23 | 97.96 |
| Diphenhydramine | 1000.000 | 9.86 | 10.50 | 8.54 |
| hydrochloride | 316.456 | 28.78 | 28.10 | 23.10 |
| | 100.144 | 85.78 | 94.55 | 70.08 |
| | 31.691 | 80.44 | 97.16 | 94.77 |
| | 10.029 | 111.50 | 102.62 | 83.05 |
| | 3.174 | 77.37 | 85.78 | 88.63 |
| | 1.004 | 97.50 | 89.08 | 86.24 |
| | 0.318 | 88.06 | 91.59 | 113.55 |
| Remdesivir | 12.000 | 65.24 | 74.83 | 80.32 |
| | 3.797 | 74.95 | 73.89 | 84.18 |
| | 1.202 | 79.39 | 77.05 | 88.39 |
| | 0.380 | 88.04 | 87.92 | 91.20 |
| | 0.120 | 93.07 | 89.44 | 88.74 |
| | 0.038 | 84.42 | 87.81 | 78.34 |
| | 0.012 | 63.61 | 115.17 | 89.44 |
| | 0.004 | 77.05 | 106.28 | 106.87 |

Although the invention has been described above in relation to preferred embodiments thereof, it will be understood by those skilled in the art that variations and modifications can be accomplished in these preferred embodiments without departing from the scope and spirit of the invention.

1. A composition comprising N,N-dimethyl-2-((methyldiphenylsilyl)oxy)ethan-1-amine.

2. A pharmaceutical composition comprising the composition of claim 1.

3. A composition comprising a compound of formula I:

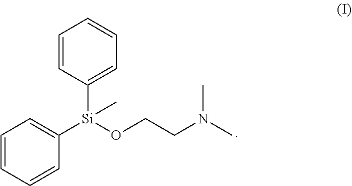 [SMILES: CN(C)CCO[Si](C)(c1ccccc1)c1ccccc1]

4. A pharmaceutical composition comprising the composition of claim 3.

5. A method of limiting the occurrence or severity of infection from viruses comprising administering a therapeutically effective amount of a N,N-dimethyl-2-((methyldiphenylsilyl)oxy)ethan-1-amine to a patient in need of treatment therefrom.

6. The method of claim 5, wherein the infection is caused by viruses that stimulate a cytokine storm.

7. The method of claim 6, wherein the viruses are selected from the group consisting of SARS-CoV-2, Ebola, Dengue, Influenza, and Marburg.

8. A method of limiting the occurrence or severity of infection from viruses comprising administering a therapeutically effective amount of the composition of claim 3 to a patient in need of treatment therefrom.

9. The method of claim 8, wherein the infection is caused by viruses that stimulate a cytokine storm.

10. The method of claim 9, wherein the viruses are selected from the group consisting of SARS-CoV-2, Ebola, Dengue, Influenza, and Marburg.

11. The method of claim 9, wherein the composition downregulates a cytokine immune response to the virus.

12. The method of claim 6, wherein the composition downregulates a cytokine immune response to the virus.
